(19) United States
(12) Patent Application Publication
(10) Pub. No.: US 2008/0161749 A1
(43) Pub. Date: Jul. 3, 2008

(54) PORTABLE INFECTION CONTROL DEVICE

(76) Inventors: Edward Houghton-Ward, Banora Point (AU); Joseph F. Boatman, Boulder, CO (US); Raymond E. Stidd, Bloomington, IN (US); Jan Gilbert, Muncie, IN (US); Jason McCrocklin, Anderson, IN (US)

Correspondence Address: ANDERSON, LEVINE & LINTEL L.L.P., 14785 PRESTON ROAD, SUITE 650, DALLAS, TX, 75254, US

(21) Appl. No.: 11/857,685

(22) Filed: Sep. 19, 2007

Related U.S. Application Data
(60) Provisional application No. 60/826,192, filed on Sep. 19, 2006.

(51) Int. Cl. A61M 35/00

(52) U.S. Cl. 604/23

(57) ABSTRACT

A sterilized environment is provided for interacting with an open wound by directing a flow of dense, sterilized gas to an open tissue site and holding the dense sterilized gas within the vicinity of the open tissue site using a dam. The gas may be densified by cooling the gas relative to the ambient temperature. A portable unit filters un-sterile gas with a HEPA or other high efficiency filter. The filtered gas is cooled and further purified with a UVC lamp.

### CROSS-REFERENCE TO RELATED APPLICATIONS

This application claims the benefit of the filing date of copending provisional application U.S. Ser. No. 60/826,192, filed Sep. 19, 2006, entitled “PORTABLE INFECTION CONTROL DEVICE”, which is incorporated by reference herein.

### STATEMENT OF FEDERALLY SPONSORED RESEARCH OR DEVELOPMENT

Not Applicable

### BACKGROUND OF THE INVENTION

1. Technical Field

This invention relates in general to medical equipment and, more particularly, to a device for providing infection control during medical procedures.

2. Description of the Related Art

Before, during and after a surgical procedure, maintaining the sanitary conditions in and around the open tissue site is of utmost importance. Naturally, all implements used in a surgical procedure are sterilized beforehand. Before, during and after the procedure, the open tissue site may become infected due to airborne microbial organisms. These organisms can cause the patient to contract serious infections before, during and after the procedure.

Research has shown that approximately 80% of viruses are airborne. However, an estimated 87% of medical facilities within the United States do not utilize advanced air filtration or ultraviolet light technology in the air ducts to kill airborne microbials.

Even in medical facilities with advanced filtration, the high rates of air exchanges necessary to reduce airborne infection rates in the healthcare environment contribute to the problem of hospital-acquired (nosocomial) infections. The medical personnel and equipment in the room carry mold spores, bacteria, and viruses on them. These contaminants are not filtered or sterilized by any existing infection control systems. They can easily fall into an open wound and infect the patient.

With the rise of antibiotic-resistant organisms, there is a more pronounced need for the increased protection of open tissue at all stages of the surgical procedure.

Therefore, a need has arisen for an improved device that can reliably maintain a sterile environment at an open tissue site.

### BRIEF SUMMARY OF THE INVENTION

In the present invention, a sterilized environment is provided for interacting with an open wound by generating a flow of a dense, sterilized gas to an open tissue site and directing the dense sterilized gas to the vicinity of the open tissue site.

### BRIEF DESCRIPTION OF THE SEVERAL VIEWS OF THE DRAWINGS

For a more complete understanding of the present invention, and the advantages thereof, reference is now made to the following descriptions taken in conjunction with the accompanying drawings, in which:

FIG. 1 illustrates a portable infection control device used in the setting of a surgical procedure;

FIG. 2 illustrates a block diagram of an embodiment of the cooling/sterilization unit of the portable infection control device of FIG. 1;

FIG. 3 illustrates a perspective view of an embodiment of the portable infection control device as attached to an IV stand;

FIG. 4 illustrates a detailed view of an embodiment of a dam of the portable infection control device of FIG. 3;

FIG. 5 illustrates an embodiment of the cooling/sterilization unit device of FIG. 2;

FIG. 6 illustrates another embodiment of the cooling/sterilization unit of FIG. 2.

### DETAILED DESCRIPTION OF THE INVENTION

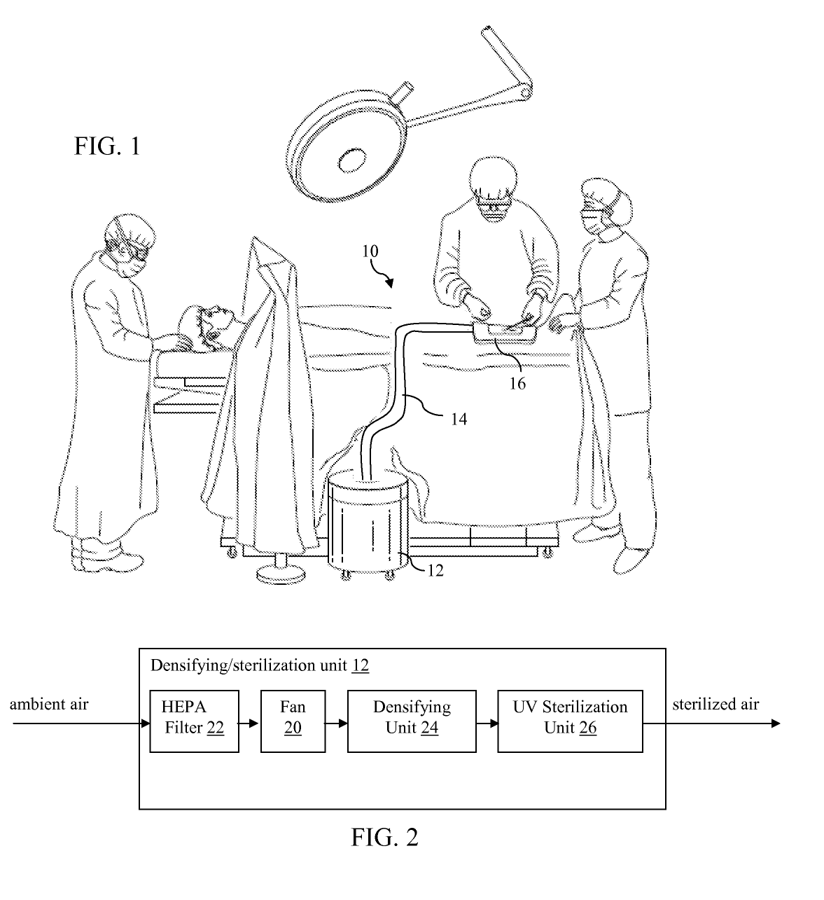

The present invention is best understood in relation to FIGS. 1-6 of the drawings, like numerals being used for like elements of the various drawings.

FIG. 1 illustrates perspective views of an infection control device 10 in operation during a surgical procedure. Infection control device 10 includes a densifying/sterilization unit 12, a hose 14, and a dam 16.

In operation, portable infection control device 10 emits a flow of a cool, sterilized gas about an open tissue site during a surgical procedure or during ward dressing. The infection control device 10 provides a blanket of sterilized gas around the open tissue site which displaces ambient, un-sterilized air around the tissue site. The un-sterilized air could allow airborne microbials to infect the open tissue.

In operation, the open tissue site is surrounded by a dam 16 encircling the open tissue site. The output of the densifying/sterilization unit 12 through hose 14 and dam 16 is a sterilized gas directed to the interior of the dam 16. The sterilized gas has been conditioned (if needed) to have a higher density than the ambient air surrounding the open tissue site. The densification of the gas could be accomplished by several means including cooling air, or another gas, or by mixing a denser, inert gas, such as carbon dioxide, with air, or another gas. One preferred method of producing a denser gas is to cool air to a temperature approximately 3-8 degrees (Fahrenheit) below that of the ambient air around the open tissue site. Since cool air has a higher density than warmer air, the cooled, sterilized air displaces the ambient air within the dam 16. The walls of the dam 16 help contain the cooled, sterilized gas in the vicinity of the open tissue site, which ideally forms a dome around the open tissue site. The gas within the dam is continuously replaced by sterilized gas from the cooling/sterilization unit 12.

A slow, constant flow of gas from the infection control device ensures that the gas surrounding the open tissue site will always be sterile. The flow rate of the gas from the densifying/sterilization unit 12 depends upon several factors, including the size of the area encompassed by dam 16. The gas flow rate should be sufficient to reach the open tissue site, but not so great that the gas flow causes turbulence sufficient to draw in unsterilized air to the open tissue site.

In the preferred embodiment, a newly sterilized hose 14 and dam 16 will be used for each procedure, since condensate may form in the hose 14 or dam 16 when the unit is not active, providing a breeding ground for microorganisms.

FIG. 2 illustrates a block diagram of an embodiment of the densifying/sterilization unit 12. A fan 20 draws an un-sterilized gas, such as ambient air, from the outside of the unit 12 through a high efficiency filter, such as a HEPA filter 22. A HEPA filter removes 99.97% (or higher) of entrained particles 0.3 micrometers in diameter from the gas. Particles larger or smaller than 0.3 are filtered at an even higher efficiency. Densifying unit 24 increases the density of the incoming gas. In an embodiment where cooling is used to increase the density of the gas, the densifying/sterilization unit 12 cools the gas to about three to eight degrees Fahrenheit below that of the ambient air. Since the densifying unit 24 only needs to reduce the temperature of the gas by a small amount, the cooling unit can be made very small. Since the flow rate is low, the fan 20, filter 22 and germicidal UV lamp 26 can also be small.

In an alternative embodiment, the density of the incoming gas is increased by mixing the incoming gas with another gas in the densifying unit 24.

The UV sterilization unit 26 kills any microbial organisms remaining in the flow of gas through the densifying/sterilization unit 12 after filtering. The UV sterilization unit 26 may use a single UVC lamp, providing the flow of gas through the unit is relatively low. Additional lamps can be used for a higher rate of flow; however, as described below, only a very low flow rate is needed because the UV sterilization unit 26 is simply displacing a small amount of un-sterilized air from around the site of an open wound.

The order of the flow through the components of the densifying/sterilization unit 12 could be varied. Fan 20 and HEPA filter 22 could be switched in order, as could densifying unit 24 and sterilization unit 26. In the embodiment shown below, the UV sterilization unit 26 is placed between cooling units, such that the gas is cooled before and after sterilization.

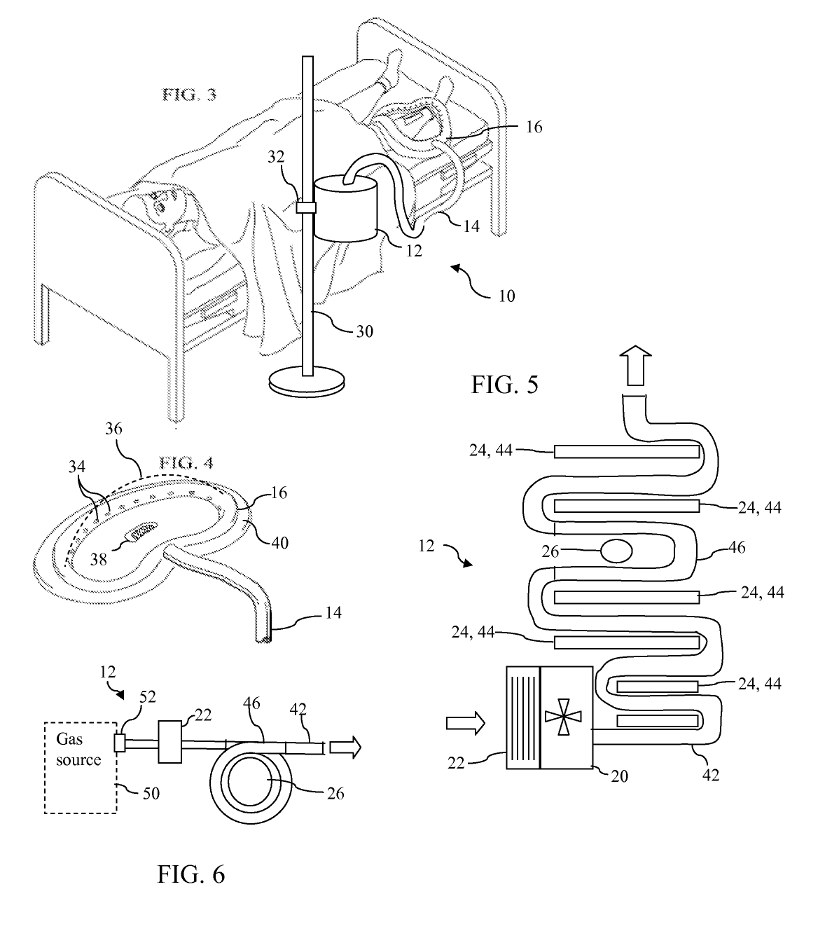

FIG. 3 illustrates an embodiment of the present invention, where the densifying/sterilization unit 12 is coupled to an IV stand 30, or other stand, using mounting clamp 32. In the preferred embodiment, the densifying/sterilization unit 12 is held above the floor, where there may be an excess of contaminants, which could be drawn into the densifying/sterilization unit 12 and prematurely clog filter 22. Clamp 32 could be used to mount the unit 12 to any above ground support.

FIG. 4 illustrates a close up perspective view of the dam 16 and hose 14, where the densified and sterilized gas is emitted from holes 34 in the dam 16 to provide a dome 36 of sterilized gas to the open tissue site 38.

The size of the dam 16 and hose 14 can vary, but it has been found that a ½″ diameter is sufficient for either dam 16 or hose 14. Raising the height of the dam will increase the volume of densified sterilized gas in the dome 36; however, a dome that is too high can interfere with access to the open tissue site 38. The dam 16 could be available in pre-made shapes, such as circular, oval and rectangular dams of different dimensions, to surround the open tissue site 38, or it could be linear with the ability to wrap around the open tissue site in a desired shape. The flow rate can be made variable to accommodate different shapes and sizes. Preferably, the dam 16 is disposable, for reasons stated above. The dam 16 may be connected to a heavier, stable material, such as a bean-bag layer 40, to hold the dam 16 in place during interaction with the open tissue site 38.

The present invention as shown above can be used in many different environments, because it directs the sterilized gas to the open tissue site and displaces un-sterile air, rather than trying to achieve sterility by sterilizing all of the air in the room. Accordingly, the present invention may be used to reduce nosocomial infections in the other medical settings, where high levels of sterility are not possible because of the coming and going of non-sterile visitors.

When drenching the wound area with sterile gas from the portable infection control device 10, hospital screens can be used to assist in reducing intrusive ambient air currents in the room. The air turbulence might otherwise mix unsterile air with the sterile gas from the portable infection control device 10, thereby reducing the effectiveness of the portable infection control device 10.

FIG. 5 illustrates an embodiment for the densifying/sterilization unit 12. A gas is drawn through filter 22 into an internal hose 42 by fan 20. The hose keeps the gas being sterilized separate from other components of the densifying/sterilization unit 12. As the gas travels through hose 42, it is cooled by a number of cooling shelves 44 containing thermo-electric coolers. A transparent section 46 of hose 42 provides exposure to the radiation from the UV sterilization unit 26. The transparent section 46 of hose 42 may encircle the UV sterilization unit 26 one or more times to increase the time that the gas inside the transparent section 46 is sterilized by the UVC radiation from the UV sterilization unit 26.

In the illustrated embodiment, the cooling shelves 44 are located on either side of the UV sterilization lamp 26. The UV sterilization unit 26 will generate some heat, which will increase the temperature of the gas while it is being sterilized. While this effect can be offset by additional cooling, it is best to provide the additional cooling after sterilization, to reduce the maximum temperature drop of the sterilized gas relative to the ambient temperature of the room. Reducing the maximum temperature drop reduces the possibility of forming condensation inside the hose 42. Other humidity removal techniques may also be used to keep the hose 42 dry.

Hose 42 may be replaceable as needed to maintain a sterile environment for the gas being conditioned.

FIG. 6 illustrates another embodiment of the densifying/sterilization unit 12, which directs a gas having a density greater than the ambient air to the open tissue site, without mixing the gas with ambient air from the room. The gas could be stored in the densifying/sterilization unit 12 in container 50, or the gas could be produced external to the densifying/sterilization unit 12; for example, the gas could be produced from an onsite generator. A regulator 52 controls the flow rate of the gas. The gas is filtered through HEPA filter 22 and is exposed to germicidal UV radiation from UV sterilization lamp 26 through transparent section 46 of hose 42.

The present invention provides significant advantages over the prior art. First, the present invention provides sterility while in un-sterile environments, such as a general surgical ward, an ambulance, a patient room, a medical office, or a recovery room. It will also increase sterility in a more or less sterile environment, such as an operating room. Second, gas is recirculated locally, so un-sterile air is not drawn into the room. Third, the present invention can reduce or eliminate the need for encapsulated suits.

Although the Detailed Description of the invention has been directed to certain exemplary embodiments, various modifications of these embodiments, as well as alternative embodiments, will be suggested to those skilled in the art. The invention encompasses any modifications or alternative embodiments that fall within the scope of the Claims.

1. A portable infection control device, comprising:
a portable unit for producing a flow of sterilized gas having a density greater than ambient air to an open tissue site;
a dam for holding the sterilized gas within the vicinity of the open tissue site.

2. The portable infection control device of claim 1 and further comprising a hose for transferring gas from the portable unit to the open tissue site.

3. The portable infection control device of claim 2, wherein the dam is coupled to the hose, such that sterilized gas from the hose is emitted from the dam to the open tissue site.

4. The portable infection control device of claim 2, wherein the sterilized gas is at least three degrees Fahrenheit below the ambient temperature.

5. The portable infection control device of claim 1 and further comprising a weight attached to the dam to hold the dam in place.

6. The portable infection control device of claim 1 wherein the portable unit comprises:
a fan;
a high efficiency filter; and
a cooling device.

7. The portable infection control device of claim 6 wherein the portable unit further comprises a UV lamp.

8. The portable infection control device of claim 7 wherein the cooling device includes cooling elements before and after the UV lamp.

9. The portable infection control device of claim 7 wherein the gas sterilized by the UV lamp flows through a tube, a portion of which is transparent to allow transmission of UV radiation to the gas.

10. A method of providing a sterilized environment for interacting with an open wound, comprising the steps of:
generating a flow of sterilized gas having a density greater than ambient air to an open tissue site; and
directing the sterilized gas to the vicinity of the open tissue site.

11. The method of claim 10 and further comprising the step of holding the sterilized gas in a dam encompassing the open tissue site.

12. The method of claim 11, wherein the directing step comprising the step of emitting sterilized gas from the dam to the open tissue site.

13. The method of claim 11 and further comprising the step of attaching a weight to the dam to hold the dam in place.

14. The method of claim 10 wherein the generating step includes the step of cooling the gas.

15. The method of claim 14 wherein the generating step comprises the step of cooling unsterilized gas, sterilizing the gas, and cooling the gas after sterilizing.

16. The method of claim 14, wherein the sterilized gas is at least three degrees Fahrenheit below an ambient temperature.

17. The method of claim 10 wherein the generating step comprises the step of filtering un-sterile gas with a high efficiency filter.

18. The method of claim 17 wherein the generating step further comprises the step of irradiating gas from the high efficiency filter with a UV lamp.

19. The method of claim 18 wherein the gas sterilized by the UV lamp flows through a tube, a portion of which is transparent to allow transmission of UV radiation to the gas.
